(19) United States
(12) Patent Application Publication
(10) Pub. No.: US 2026/0345480 A1
(43) Pub. Date: Nov. 5, 2026

(54) PHARMACEUTICAL DOSAGE FORMS FOR PULSATILE RELEASE

(71) Applicant: CHIESI FARMACEUTICI S.P.A., Parma (IT)

(72) Inventors: Marisa PERTILE, Parma (IT); Matteo CEREA, Parma (IT); Anastasia FOPPOLI, Parma (IT); Andrea GAZZANIGA, Parma (IT); Saliha MOUTAHARRIK, Parma (IT)

(73) Assignee: CHIESI FARMACEUTICI S.P.A., Parma (IT)

(21) Appl. No.: 19/487,159

(22) PCT Filed: May 21, 2024
(86) PCT No.: PCT/EP2024/063991

§ 371 Date: Nov. 24, 2025

(30) Foreign Application Priority Data
May 25, 2023 (EP) 23175449.0

(51) Int. Cl. A61K 9/00; A61K 9/28; A61K 31/4412; A61K 47/02; A61K 47/12; A61K 47/24; A61K 47/34; A61K 47/36

(52) U.S. Cl. CPC A61K 9/0002; A61K 9/2866; A61K 9/2886; A61K 31/4412; A61K 47/02; A61K 47/12; A61K 47/24; A61K 47/34; A61K 47/36

(57) ABSTRACT

The invention relates to pharmaceutical dosage forms comprising a drug having a fast absorption in the gut, which is absorbed in distal intestinal tract including the colon, and for which pulsatile release is necessary to avoid the potential toxicity of the drug continuous absorption. In particular the invention is directed to a dosage form comprising a formulation in form of coated small units to deliver the active ingredient after a programmed time period following oral administration and processes of preparation thereof. In a preferred embodiment the drug is the iron chelator deferiprone.

### FIELD OF THE INVENTION

The invention relates to pharmaceutical dosage forms comprising a drug having a fast absorption in the gut, which is absorbed in distal intestinal tract including the colon, and for which pulsatile release is necessary to avoid the potential toxicity of the drug continuous absorption.

### BACKGROUND OF THE INVENTION

Among modified-release oral dosage forms, increasing interest has currently turned to systems designed to achieve time-specific (delayed, pulsatile) and site-specific delivery of drugs. In particular, systems for delayed release are meant to deliver the active principle after a programmed time period following administration (Maroni A et al., Expert Opinion on Drug Delivery, 2005, 2(5), 855-871).

These systems constitute a relatively new class of products which has started to catch on with the recent advances in chronopharmacology. It is by now well-known that the symptomatology of a large number of pathologies as well as the pharmacokinetics and pharmacodynamics of several drugs follow temporal rhythms, often resulting in circadian variations. Therefore, the possibility of exploiting delayed release to perform chronotherapy is quite appealing for those diseases, the symptoms of which recur mainly at night time or the early morning, such as bronchial asthma, angina pectoris and rheumatoid arthritis.

Oral pulsatile delivery systems with repeated release pulses (multiple pulse systems) may be taken into account to enhance patient compliance when multiple-dosing daily regimens are prescribed, but the indicated drug fails to constitute a suitable candidate for prolonged release; for example, this may concern molecules that undergo a strong first-pass effect in the liver or else develop pharmacological tolerance.

In the art, the delay in the onset of release has been achieved through osmotic mechanisms, hydrophilic or hydrophobic layers and swellable or erodible plugs sealing a drug-containing insoluble capsule body.

A considerable number and variety of both single- and multiple-unit oral pulsatile delivery systems are devised as a drug reservoir provided with an outer release-controlling water-insoluble but permeable coating subject to mechanically induced rupture phenomena. Such membrane undergoes partial or complete breakup within a programmable time period after the systems are immersed in the aqueous fluids, thus allowing the inner drug formulation to be exposed directly to the bulk medium. The film rupture responsible for drug release occurs as a consequence of an increasing outward pressure, which results from expansion of the inner core. This may in turn be attained by including swellable, osmotic or effervescent additives in the drug reservoir.

In particular, multiple-unit drug delivery systems are endowed with some biopharmaceutical advantages linked to the lower variability of the drug absorption profile.

In fact, for single-unit systems the gastric emptying time is strongly influenced by the inter-digestive or digestive phase in which administration takes place. In contrast, multiple-unit dosage forms consisting of numerous small subunits may be able, depending on their size and density, to pass the stomach even when the pylorus is contracted and spread along the gut, thus possibly reducing the gastrointestinal transit variability and, particularly when the drug absorption rate is affected by the release site, the inter- and intrasubject differences in the absorption profiles. Another advantageous is related to the subdivision of the dose in several subunits, which allows a distribution of the delivered dose on an extended surface area thus lowering the potential risk of mucosal injury caused by high local drug concentrations.

Moreover, the subdivision of the dose reduces the probability of dose dumping.

In the art, examples of platform technologies utilized for preparing multiple-unit delivery systems are Time-Controlled Explosion System also known as TES (Ueda S et al J Drug Target. 1994; 2(1):35-44; Shimono N et al. Chem Pharm Bull, 2002, September; 50(9):1169-75, and the Chronotopic™ system (Gazzaniga A et al. Eur J Pharm. Biopharm 1994, 40(4), 246-250; Sangalli, M. E.; et al. Chronotopic™ Technology. In Chronopharmaceutics: Science and Technology for Biological Rhythm-Guided Therapy and Prevention of Diseases; Youan, B.-B. C., Ed.; John Wiley and Sons Inc.: Hoboken, NJ, USA, 2009, pp 145-163).

In particular, the Chronotopic™ system was also proposed in a colon-targeting configuration, enabling the attainment of a lag phase matching the small intestinal transit time and in a pulsatile release configuration intended for bed-night administration to address early morning pathologies.

Both systems are designed as drug-containing cores, coated with one or more layers capable of defining the release kinetics of the drug.

A sketch of the TES and Chronotopic™ systems are reported in FIGS. 1 and 2, respectively.

On the other hand, said systems relies on a somewhat problematic design as their composition systems shall be fine-tuned depending on the desired release profiles (lag time and frequency of the pulses, delay between the pulses).

In particular said features are determined by the thickness and the characteristics of the coatings applied. However, we have found that the thickness of the coating often turns out to be poorly reproducible and not correlated to the duration of the process.

Furthermore, their ability to delay the onset of drug release is markedly affected by minimal changes in the thickness of the layers, which are difficult to fine-tune.

Deferiprone is a well-known iron chelator drug indicated in treatment of diseases characterized by iron accumulation.

Currently said drug is available as Ferriprox® in form of tablets for twice-a-day or three times-a-day oral administration.

Therefore, to improve the compliance of the patient, it would be advantageous to provide a formulation for once-a-day administration.

However, in developing a formulation for once-daily administration, pulsatile release is necessary to avoid the potential toxicity of deferiprone observed in animals undergoing continuous infusion for periods longer than 24 hours. In other words, the development of an extended-release formulation is not recommended.

Furthermore, deferiprone is a drug for which high and personalized doses are required according to the weight of the patient.

Therefore, it would be advantageous to provide a formulation for pulsatile release able of delivering deferiprone after a programmed time period.

In particular, in view of the potential drawbacks of the platform technologies of the prior art, it would be highly advantageous to provide a robust and versatile multiple unit dosage forms capable of determining a pulsatile release of the drug, in particular deferiprone.

### SUMMARY OF THE INVENTION

In a first aspect, the invention relates to a pharmaceutical dosage form comprising coated small units, wherein each unit in turn comprises an active ingredient, a filler, a lubricant and/or a glidant and it is coated with an inner layer and, optionally an outer layer, whereby one part of the coated small units releases a fraction of the active ingredient in a time comprised between 10 minutes and 8 hours, while the remaining part releases the remaining active ingredient. Said dosage form is suitable for providing a pulsatile release profile.

Preferably, at least the coated small units releasing fraction of the active ingredient in a time comprised between 10 minutes and 8 hours are covered with the outer layer.

More preferably at least the part of the small units releasing the active ingredients in a time comprised between 6 hours and 24 hours are coated with the outer layer.

Advantageously, the coated small unit comprises the active ingredient in an amount comprised between 75% and 90%, a filler in an amount comprised between 10% and 24%, and a lubricant and/or glidant in an amount of 0.5 to 1.0%, all the amounts calculated by weight on the total weight of the uncoated unit;
wherein the inner layer comprises a swellable hydrophilic polymer and a plasticizer, and the outer layer, when present, comprises a film-forming insoluble polymer and an agent with channeling properties.

Preferably, the active ingredient is deferiprone.

Preferably the pharmaceutical dosage form is a capsule or a sachet.

Advantageously, the small unit to be coated is in form of minitablets or pellets.

Accordingly in a second aspect the invention provides a process for the preparation of the coated minitablets, said process comprising the following steps:
(i) mixing the active ingredient with the filler agent, and with the lubricant/glidant excipient to form a mixture;
(ii) compressing the mixture obtained in step (i) to form the minitablets;
(iii) coating the minitablets with the inner layer, and
(iv) optionally, coating the minitablets of step iii), with a further outer layer to obtain double coated minitablets, and
(v) drying the coated minitablets.

Optionally, the mixture of step i) is wet granulated in order to obtain granules suitable to be tableted.

In an alternative embodiment, when the small coated units are in form of pellets, the invention provides a process for the preparation of the coated pellets, said process comprising the following steps:
(i) mixing the active ingredient with the filler agent, and with the lubricant/glidant excipient to form a mixture;
(ii) wetting the mixture of step i) with a suitable liquid binder;
(iii) extruding the wet mass of step ii) through a die with holes to form cylindrical extrudates;
(iv) spheronizing the extudates of step iii) to obtain wet pellets;
(v) drying the obtained pellets:
(vi) coating the pellets with the inner layer;
(vii) optionally coating the pellets of step vi) with a further outer layer, and
(viii) drying the coated pellets.

In a third aspect, the invention is directed to a process for filling the pharmaceutical dosage form with the coated small units according to the invention.

In a fourth aspect, the invention is directed to the claimed pharmaceutical dosage form comprising deferiprone as active ingredient for use for the treatment of diseases which cause an overload of iron, or for the prevention and/or treatment of diseases which are caused by an overload of iron.

In a fifth aspect, the invention is directed to the claimed pharmaceutical dosage form comprising deferiprone as active ingredient in the manufacture of a medicament for the treatment of diseases which cause an overload of iron, or for the prevention and/or treatment of diseases which are caused by an overload of iron.

In a sixth aspect, the invention refers to a method for the treatment of diseases which cause an overload of iron, or for the prevention and/or treatment of diseases which are caused by an overload of iron thereof in a patient in a need thereof, said method comprising orally administering the claimed pharmaceutical dosage form comprising deferiprone as active ingredient.

In a seventh aspect, the invention is directed to a method for reducing gastric distress or the risk of gastric distress in a patient in need of deferiprone treatment, comprising orally administering to the patient the claimed dosage form.

### BRIEF DESCRIPTION OF THE DRAWINGS

FIG. 1—Sketch of the structure of T.E.S. system consisting of nonpareil seeds over which drug is stratified, provided with a double layer coating: a inner swelling layer (L-HPC, low substituted hydroxy propyl cellulose) and an outer water insoluble polymeric membrane (ethylcellulose, EC).

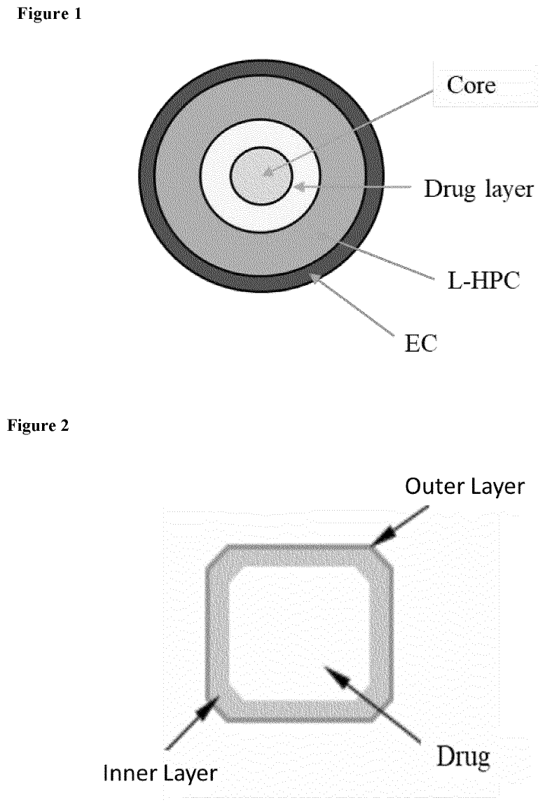

FIG. 2—Sketch of the structure of Chronotopic system.

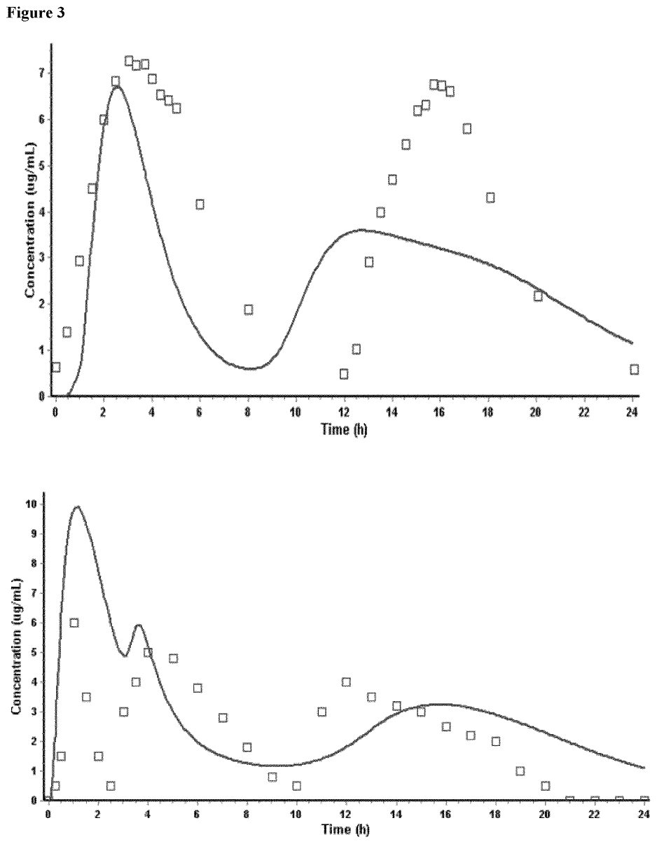

FIG. 3—Alternative plasma concentration versus time profile simulated with the validated PBPK model (solid line); desired in-silico generated profiles (squares line).

FIG. 4—Release profiles of deferiprone from mini-tablets obtained according to the invention; a) C0, C3, C6; b) C9, C11, C17.

FIG. 5—Relationship between lag time and thickness of the outer layer.

### DEFINITIONS

As used herein, the indefinite articles “a” or “an” should be understood to refer to “one or more” of any recited or enumerated component. For example, “a tablet” refers to one or more tablets.

Also as used herein, “and/or” refers to and encompasses all possible combinations of one or more of the associated listed items, as well as the lack of combinations when interpreted in the alternative (“or”).

When the term “about” is used in conjunction with a numerical value or range, it modifies that value or range by extending the boundaries above and below the numerical values set forth. The term “about” is used herein to modify a numerical value above and below the stated value by a variance of 10 percent, up or down (higher or lower), i.e., ±10%, unless a different variance is indicated (e.g., ±30%, ±20%, ±5%, ±1%, etc.).

Wherever aspects are described herein with the language “comprising,” otherwise analogous aspects described in terms of “consisting of and/or” consisting essentially of are also provided. To the extent that the term “includes” or “including” is used in the specification or the claims, it is intended to be inclusive in a manner similar to the term “comprising” as that term is interpreted when employed as a transitional word in a claim.

As used herein, the term “active ingredient” or “active pharmaceutical ingredient” (API) or “drug” are used as synonymous and mean any component that is intended to furnish pharmacological activity or other direct effect in the diagnosis, cure, mitigation, treatment, or prevention of disease, or to affect the structure or any function of the body of man or other animals.

The terms “iron overload” or “overload of iron” are used interchangeably herein and refer to medical conditions where the body contains or stores too much (or “excess”) iron. An example is transfusional iron overload, where the excess iron is introduced by one or more blood transfusions.

The term “coated small units” refer to pharmaceutical forms comprising an active ingredient, and one or more excipients having a diameter of few millimeters coated with at least one layer.

The term “minitablets” commonly refers to compressed tablets with size smaller than typical tablets. Although there are currently no regulatory guidelines defining minitablets (sometimes referred to as microtablets), the term has been used to describe tablets with diameters between 0.3 to three millimeters.

The term “pellets” refers to pharmaceutical forms formed by the agglomeration of fine powdered excipient and an active ingredients together that leads to the formation of small free flowing spherical or semi spherical particles.

In the present context, the term “hydrophilic” describes a molecule or portion of a molecule which is typically electrically polarized and capable of forming hydrogen bonds with water molecules, enabling it dissolve more readily in water than in oil or other “non-polar” solvents.

Conversely, the term “hydrophobic” denotes a compound tending to be electrically neutral and non-polar, and thus preferring other neutral and nonpolar solvents or molecular environments.

For “pulsatile drug release” it is meant the repeated succession of drug pulses at variable time intervals.

For “with channeling properties” it is intended an excipient with swelling properties in water capable of favoring liquid inflow by generating molecular-to-micron size channel/pores in the film of a film-forming insoluble polymer.

“Lag time” in pharmacokinetics (PK) corresponds to the finite time taken for a drug to appear in systemic circulation following extravascular administration. Lag time is a reflection of the processes associated with the absorption phase such as drug dissolution and/or release from the delivery system and drug migration to the absorbing surface.

For “pH dependent solubility” it is meant a substance having different solubilities at different pHs. These pH-dependent solubility differences lead to pH-dependent dissolution profiles.

The expression “insoluble or poorly water soluble” refers to a substance having a solubility in water as defined in the European Pharmacopoeia Ed. 4^{th}, 2003, page 2891.

In the present context, the terms “controlled release”, “prolonged release”, “modified release” and “delayed release” are collectivley indicated as “DR” and are intended to be terms covering some types of release of deferiprone from a composition of the invention that is appropriate to obtain a specific therapeutic or prophylactic response after administration to a subject.

“Superdisintegrant agent” as used herein refers to an excipient that is insoluble in water, but swells when wetted to cause a tablet to disintegrate.

“Dissolution” as used herein refers to the process by which a solute forms a solution in a solvent.

“Enteric coat” or “enteric coating” as used herein refers to a coating comprising an enteric polymer. An enteric coating can serve to prevent or delay a tablet's dissolution or disintegration in a gastric environment.

“Enteric coated tablet” means a tablet having a core comprising an active ingredient, which is coated with an enteric coating.

“Enteric polymer” as used herein is understood to mean a polymer that is relatively insoluble at the acidic pH of the fasted stomach (e.g., about pH 1 to about pH 4), but soluble at higher pH (e.g., about pH 4.5 to about pH 8), which corresponds to the pH in the small intestine or thereafter, particularly in the duodenum or ileum.

The term “plasticizer” means an additive that increase the elasticity of coatings based on film-forming material.

The term “filler” means a pharmacologically-inert pharmaceutically acceptable excipient used to make a drug product easier to handle.

The terms “diluents” and “bulking agents” are used as synonymous.

With the term “therapeutic equivalence” is meant a drug product having the same clinical effect and safety profile of a reference product when administered to patients

With the term “PK bioequivalence” it is meant the absence of a significant difference between the bioavailability, i.e., the extent of absorption and peak concentration, between two pharmaceutical drug products (e.g., a test product and a reference product) over the course of a period of time, at the same dose and under the same conditions.

The determination of whether or not a test product is PK bioequivalent to a reference product is determined by performing a study, referred to as a bioequivalence or comparative bioavailability study, in a group of subjects, usually about 18-36 subjects or more, under controlled conditions.

The PK study can be done in a “crossover” design, which means that the study is done in 2 or more phases, usually at least a week apart, depending in part on the half-life of the drug. In the first phase, half the subjects are randomly assigned to ingest the test product first and the other half ingest the reference product first. In the second phase, each subject ingests the alternate product.

In each phase, blood samples are drawn from each subject, on a predetermined schedule after ingestion of the test product. The blood samples are then analyzed to determine serum concentrations of the drug at each time point. For example, drugs are bioequivalent if they enter circulation at the same rate when given in similar doses under similar conditions. Parameters often used in bioequivalence studies are tₘₐₓ, Cₘₐₓ, Cₘᵢₙ, AUC_{0-infinity}, AUC₀₋ₜ.

In the present context “tₘₐₓ” denotes the time to reach the maximal plasma concentration (Cₘₐₓ) after administration; “AUC_{0-infinity}” denotes the area under the plasma concentration versus time curve from time 0 to infinity; “AUC₀₋ₜ” denotes the area under the plasma concentration versus time curve from time 0 to time t; “W50” denotes the time where the plasma concentration is 50% or more of Cₘₐₓ; “W75” denotes the time where the plasma concentration is 75% or more of Cₘₐₓ; and “MRT” denotes mean residence time for the active ingredient.

“Fasted state” as used herein refers to abstinence from food for a defined period of time after a meal (typically, at least several hours, e.g., 4 or 6 hours, after a meal).

“Fed state” as used herein refers to administration with a meal or soon after a meal (e.g., within about 1 hour).

The term “chemical stable” refers to stability of the active agent in the formulation, wherein changes in the drug assay values and/or impurities content are equal to or lesser than 5%, preferably lesser than 3%, during storage at 25 °C and 60% relative humidity (RH), or 40 °C and/or 75% RH, for at least 1 month.

The term “in vitro-in vivo” correlation (IVIVC) refers to an in vitro dissolution test that is predictive of the in vivo performance of the drug product.

“Gastric distress” as used herein refers to discomfort of the gastrointestinal (GI) tract, e.g., one or more of pain, cramping, bloating, nausea, indigestion, heartburn, and gas.

“Percent” or “%” as used herein refers to weight percentage (w/w) unless otherwise specified.

Terms such as “treating” or “treatment” or “to treat” or “ameliorating” or “alleviating” or “to alleviate” can refer to both 1) therapeutic measures that cure, slow down, lessen symptoms of, reverse, and/or halt progression of a diagnosed pathologic condition or disorder and 2) prophylactic or preventative measures that prevent, reduce the incidence of, reduce the risk of, and/or slow the development of a targeted pathologic condition or disorder. Thus, those in need of treatment include those who already have the disorder; those prone to developing the disorder; and those in whom the disorder is to be prevented. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those who already have the condition or disorder as well as those prone to developing the condition or disorder or those in which the condition or disorder is to be prevented or incidence reduced.

By “subject” or “individual” or “patient,” is meant any human subject, for whom diagnosis, prognosis, treatment, or therapy is desired.

By “therapeutically effective dose or amount” or “effective amount” is intended an amount of active pharmaceutical ingredient, e.g., deferiprone, that when administered brings about a positive therapeutic response with respect to treatment or reduces the risk of a disease in a subject to be treated.

It will be understood that the deferiprone DR tablets used as the “reference” or “Reference Product” herein are the immediate release 1000 mg Ferriprox® tablets for three-times-a-day administration and the 1000 mg delayed release Ferriprox® tablets 1000 mg) as approved by FDA and sold in the United States.

In the context of the present description, the term “synergistic” means that the capacity of the two coating layers in slowing down the penetration of water is more than would be expected by the capacity of each layer alone.

### DETAILED DESCRIPTION OF THE INVENTION

The present invention concerns pharmaceutical formulations providing a pulsatile release of an active ingredient, in particular deferiprone.

Since it has been established that for Ferriprox® tablets, that deferiprone modified release formulations exhibit a good IVIV correlation (WO 2019/082128), it is contemplated that the in vitro release profile will reflect the in vivo behaviour.

Therefore, to support the development of deferiprone pulsatile formulation in-silico deconvoluted in-vitro dissolution profiles were generated that could match the desired plasma versus time profile.

A physiological-based pharmacokinetic (PBPK) model was utilized.

Simulations were performed using the advanced compartmental absorption and transit model (ACAT) model implemented in the GastroPlus™ version 9.8 (Simulations Plus Inc, USA) based on the available clinical data (Kushvah V et al Pharmaceutics 2021 February; 13(2): 283).

To understand the influence of different release profiles, various in-vitro dissolution profiles were simulated in-silico and fitted to a Weibull distribution, an algorithm well-known to the skilled person in the art.

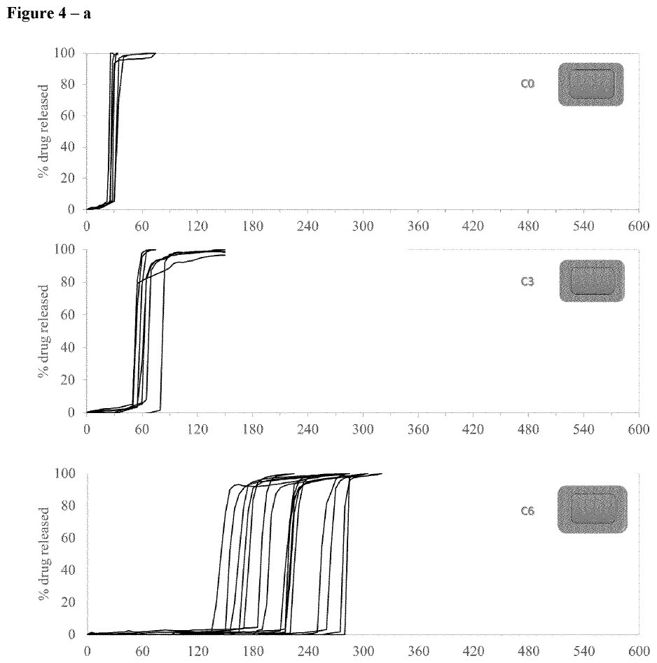

Possible suitable profiles are reported in FIG. 3. All suitable profile exhibit a lag time of about half an hour, while one or two peaks between 30 minutes and 6 hours, and a further peak between 8 and 24 hours.

The PK parameters when two or three peaks are expected are reported in Tables A and B, respectively.

TABLE A
PK parameters of the plasma concentration profile when two peaks are expected.

| Parameter | #2_0 | #2_0_4000 | #2_1 | #2_2 | #2_3 | #2_4 |
|---|---|---|---|---|---|---|
| Fa (%) | 65.551 | 61.144 | 77.594 | 74.013 | 71.797 | 77.993 |
| F (%) | 54.500 | 50.648 | 65.182 | 61.698 | 59.791 | 65.119 |
| Cmax (ug/mL) | 6.703 | 8.937 | 11.484 | 6.706 | 5.369 | 9.3457 |
| tmax (h) | 2.560 | 2.560 | 2.560 | 2.560 | 2.560 | 2.56 |
| AUC0-∞ (ug · h/mL) | 67.164 | 86.454 | 75.322 | 74.786 | 72.923 | 78.158 |
| AUC0-24 h (ug · h/mL) | 61.560 | 75.788 | 71.761 | 70.171 | 67.862 | 74.308 |
| Dose (mg) | 3000 | 4000 | 3000 | 3000 | 3000 | 3000 |

Fa: cumulative % intestinal absorption, F: % bioavailable, Cmax: maximum concentration, tmax; time to reach maximum concentration, AUC0-∞: area under the curve from time 0 to infinite, AUC0-24 h: area under the curve from time 0 to 24 h.

| Parameter | #3_0 | #3_1 | #3_2 | #3_3 |
|---|---|---|---|---|
| Fa (%) | 77.988 | 76.306 | 72.344 | 74.153 |
| F (%) | 65.23 | 63.798 | 60.34 | 61.764 |
| Cmax (ug/mL) | 9.9231 | 8.0276 | 10.356 | 9.8889 |
| tmax (h) | 1.2 | 1.2 | 1.36 | 1.12 |
| AUC0-∞ (ug · h/mL) | 77.405 | 75.89 | 72.83 | 75.224 |
| AUC0-24 h (ug · h/mL) | 74.676 | 72.974 | 68.622 | 70.028 |

Therefore in a first aspect, the invention relates to a pharmaceutical dosage form providing a pulsatile release profile, said form comprising coated small units, wherein each unit in turn comprises an active ingredient, a filler agent, a lubricant and/or a glidant and it is coated with an inner layer and, optionally an outer layer, whereby one part of the coated small units releases part of the active ingredient in a time comprised between 10 minutes and 8 hours, while the remaining part of the coated small units releases the remaining part of the active ingredient, preferably, in a time comprised between 6 hours and 24 hours.

Preferably, at least the coated small units releasing fraction of the active ingredient in a time comprised between 10 minutes and 8 hours are covered with the outer layer.

More preferably at least the part of the small units releasing the active ingredients in a time comprised between 6 hours and 24 hours are coated with the outer layer.

For example, one part of the coated small units could release part of the active ingredient in a time comprised between 30 minutes and 6 hours, while the remaining part of the coated small units could release the remaining part of the active ingredient in a time comprised between 8 hours and 24 hours.

The first part of the release pulse could occur in one to three peaks, preferably one or two peaks, while the second part of the release pulse typically gives rise to one peak only.

Advantageously, the part of the coated small units releasing the active ingredient in a time comprised between 10 minutes and 8 hours could be comprised between 20 and 80%, preferably between 30 and 70% by weight. In certain embodiments it could be comprised between 40 and 60% by weight.

Analogously, the fraction of the active ingredient released in a time comprised between 10 minutes and 8 hours could be comprised between 20 and 80%, preferably between 30 and 70% by weight. In certain embodiments it could be comprised between 40 and 60% by weight.

Advantageously the in vivo lag time, if present, is equal to or lesser than one hour, preferably between 2 and 50 minutes, more preferably of 30-45 minutes.

Advantageously the coated small unit comprises the active ingredient in an amount comprised between 75% and 90%, a filler in an amount comprised between 10% and 24%, a lubricant and/or glidant in an amount of 0.5 to 1.0%, all the amounts calculated by weight on the total weight of the uncoated unit, and it is coated with an inner layer and optionally an outer layer, wherein the inner layer comprises a swellable hydrophilic polymer and a plasticizer, and the outer layer, when present, comprises a film-forming insoluble polymer and an agent with channeling properties.

The pharmaceutical dosage form of the invention may comprise any drug having a fast absorption in the gut, which is absorbed in distal intestinal tract including the colon, and for which pulsatile release is necessary to avoid the potential toxicity of the drug continuous absorption. Examples of these active ingredients might be represented by methylphenidate HCl, amoxicillin and deferiprone.

Preferably, the active ingredient is deferiprone.

Within the scope of the present invention, deferiprone is in any physical form (crystals, amorphous powder, any possible polymorphs, any possible solvate). Included are also pharmaceutically acceptable salts and/or solvates thereof. Preferably, deferiprone is used as a base in its anhydrous form.

It has been found that the coated small units according to the invention show satisfactory technological characteristics and release performance meeting the desired pulsatile behaviour.

In a preferred embodiment, the small unites coated with the inner layer are further coated with the outer layer.

In fact, without being limited by the theory, it is expected that when present, the outer layer would act synergistically with the inner layer to slow down the penetration of water. Therefore, the two layers together constitute a very efficient controlling mechanism responsible for the delay of the release of the drug in the bowel.

In particular, the delay is mainly correlated to the thickness of the outer layer.

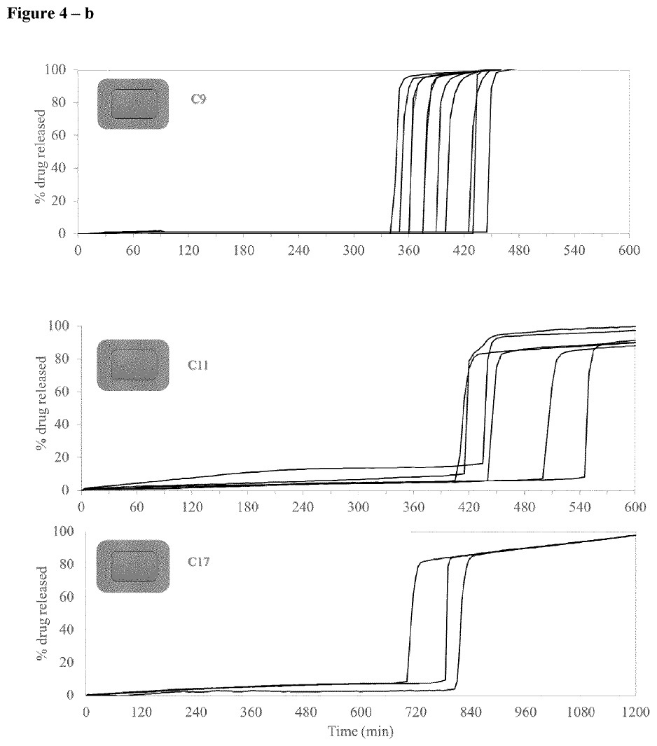

As it can be appreciated from FIG. 4, C0 and C3 coated small units displays an in vitro release profile potentially providing the first release pulse occurring in one or two peaks of the simulated in vivo profile, while C9, C11 and C17 small units provide the delayed release involved/necessary to in the second (or third) peak of the simulated profile, while C6 small units have a release profile that could match either the first part or the delayed part of the simulated in vivo profile.

In contrast with the findings of the present invention, we observed that, according to the what known in the art, the TES approach is not able to reach out the desired release performances, as the thickness of EC layer resulted poorly reproducible and not correlated to the duration of the process. In fact the ability to delay the onset of drug release is markedly affected by minimal changes in its thickness which are difficult to fine-tune.

It will be appreciated that the dosage form according to the invention could surprisingly deliver the complete dosage of the drug to achieve the desired pulsatile release of the drug over the course of about 24 hours with a single oral administration.

For deferiprone, it is also contemplated that the dosage form of the invention would turn out to be therapeutically bioequivalent to the immediate release reference Ferriprox® tablets for three-times-a-day administration and/or to the reference Ferriprox® delayed-release tablets for twice-a-day administration. Adventitiously, the coated small units are in forms of minitablets or pellets, preferably minitablets.

The filler shall be selected form the group of diluents and superdisintegrant agents or mixture thereof. Diluents could also be indicated as bulking agents.

Preferably, the filler is a mixture of a diluent and a superdisintegrant agent in any suitable ratio.

Advantageously, the diluent could be selected from the group consisting of calcium carbonate, dibasic calcium phosphate, tribasic calcium phosphate, calcium sulfate, microcrystalline cellulose, powdered cellulose, dextrans, dextrins, dextrose, fructose, kaolin, lactose, mannitol, sorbitol, starch, pregelatinized starch, sucrose, alpha-lactose monohydrate.

Preferably the diluent is microcrystalline cellulose of different varieties. For instance microcrystalline cellulose as known as Avicel PH 101 marketed by DuPont Inc (Wilmington, USA) could be used.

When the small units are in form of pellets, microcrystalline cellulose marketed as Vivapur 101 by JSR Pharma GmbH (Rosemberg, Germany), could be used, a variety especially suitable for wet granulation.

The superdisintegrant agent is selected from the group consisting of carboxymethylcelluose and its crosslinked sodium salt known as croscarmellose sodium, crospovidone (also known as cross-linked PVP), and sodium starch glycolate.

All said superdisintegrants agents are commercially available.

For example, sodium starch glycolate is sold as Explotab® CLV by JSR Pharma (Rosemberg, Germany).

More advantageously, the filler is a mixture of a diluent, preferably microcrystalline cellulose and a superdisintegrant in a ratio between 85:15 and 75:25 w/w, preferably 80:20 w/w.

Preferably, the superdisintegrant agent is in turn a mixture of crospovidone with sodium starch glycolate in a ratio of 50:50 and 30:70, preferably 35:65 by weight.

In one embodiment, when the small units are in form of pellets, a mixture of microcrystalline cellulose and carboxymethylcellulose marketed as Avicel CL 611 by DuPont Inc (Wilmington, USA) could be used.

The composition of the small unit of the invention also comprises a lubricant to prevent sticking to the tooling during compression into tablets, and/or a glidant to improve flow in the tableting process, or combinations thereof.

Advantageously, the lubricant is selected from the group consisting of magnesium stearate, calcium stearate, stearic acid, sodium stearyl fumarate, or any combination thereof.

Advantageously, the glidant is selected from the group consisting of colloidal silicon dioxide, starch and talc, preferably colloidal silicon dioxide (also known as colloidal silica) or any combination thereof.

In a preferred embodiment, the small unit comprises a mixture of colloidal silicon dioxide and magnesium stearate in a ratio of 10:90 to 20:80, preferably 15:85 by weight.

All the aforementioned excipients are commercially available.

To achieve the programmed release of the active ingredient in the gut, the small unit is suitably coated with swellable/erodible coating layers.

Advantageously they are coated with an inner layer comprising a swellable hydrophilic polymer such as a hydrophilic derivative of cellulose, for example hydroxyethylcellulose or hydropropylmethylcellulose.

More advantageously the inner layer comprises a hydroxypropyl methylcellulose and a plasticizer and optionally a glidant such as talc, in any suitable ratio by weight.

The presence of the talc makes the layer lesser sticky, improving the handling of the formulation.

The hydroxypropylmethylcellulose polymer (HPMC) of different molecular weights is commercially available from as Methocel®, for example from DuPont (Delaware, USA).

Preferably, a HPMC of low viscosity is used as it allows a more rapid efficient coating process in an aqueous medium and it is endowed with lesser gelling properties than HPMC of higher viscosity which could slow down the diffusion and hence the release of the active ingredient.

For example, Methocel® E50 could be used, a highly substituted HPMC which is reported to give a low viscosity (about 50 cP at a 2% addition in water at 20 °C).

Advantageously, the plasticizer is selected from the group comprising, but not limited to, diethyl phthalate, citrate esters such as triethyl citrate (TEC), polyethylene glycol, glycerol, acetylated glycerides, acetylated citrate esters, dibutyl sebacate, castor oil, or any combination thereof, preferably polyethylene glycol.

Polyethylene glycol (PEG) of different molecular weights is available from Sigma Aldrich Inc (St. Louis, USA). Preferably PEG 400 is used as it is reported to be endowed with better plasticizing properties.

According to a preferred embodiment, the inner layer may be constituted of HPMC and PEG 400 in a ratio comprised between 95:5 and 80:20, more preferably 90:10 by weight.

According to another preferred embodiment, the inner layer may be constituted of HPMC PEG 400, and talc in a ratio of 89:10:1.0 by weight.

The outer layer, when present, comprises a film-forming insoluble polymer and an agent with channeling properties in a suitable ratio by weight.

More advantageously said agent with channeling properties is a superdisintegrant agent such as of croscarmellose sodium, crospovidone, and sodium starch glycolate, preferably starch sodium glycolate.

Suitable film-forming insoluble polymers available on the market are: a blend of polyvinyl acetate and povidone in the ratio 8:2 sold under the trademark of Kollidon SR; methacrylate derivatives such as Eudragit RS and Eudrary RL, and cellulose derivatives such as ethylcellulose.

In a preferred embodiment of the invention, the superdisintegrant is sodium starch glycolate and the film-forming insoluble polymer is ethylcellulose.

Advantageously the ratio between the superdisintegrant agent and the film-forming insoluble polymer is comprised between 95:5 and 80:20, preferably 90:10 by weight.

As reported above, sodium starch glycolate is marketed as Explotab® CLV by JSR Pharma GmbH (Rosemberg, Germany).

Ethylcellulose (EC) is marketed as aqueous dispersion with the name of Surelease® by Colorcon Inc (PA, USA).

According to preferred embodiments, the present invention relates to coated small units in form of minitablets having the composition reported in Table 1.

TABLE 1

| Core unit | % w/w | Inner layer | % w/w | Outer layer | % w/w |
|---|---|---|---|---|---|
| Deferiprone | 81.8 | | | | |
| Avicel pH 101 | 14.4 | | | | |
| Colloidal silica | 0.1 | | | | |
| PVP | 0.8 | | | | |
| Mg stearate | 0.5 | | | | |
| Explotab ® CLV | 2.4 | | | | |
| | | Methocel E50 | 90 | | |
| | | PEG 400 | 10 | | |
| | | | | Surelease ® | 95.0 |
| | | | | Explotab ® CLV | 5.0 |

As mentioned above, thickness of the layers is of paramount importance to determine the release profile.

Therefore said thickness was determined according to different methods, ie weight gain after coating (%), coating layer thickness (μm), coating amount per unit area (mg/cm²), while the latter one being considered the most reliable and convenient one as it would be independent from the weight of the nuclei.

Detailed procedures are reported in the paragraph about the general experimental details and methods.

Advantageously, the coating thickness of the inner layer shall be comprised between about 50 and 300 micron, preferably comprised between about 60 and 280 micron, more preferably comprised between 200 and 260 micron, the latter interval corresponding to a coating amount per unit area of about 20-25 mg/cm².

In one embodiment, the thickness of the inner layer could be of about 80 micron corresponding to 6% weight increase, while in other embodiment, the thickness could be of about 200 micron corresponding to 60% weight increase.

The coating thickness of the outer layer shall be comprised between 0 and about 60 micron, corresponding to a coating amount per unit area of 0 to 10 mg/cm².

More advantageously, to make coated small units suitable for the first phase of the release, the coating outer layer could be present or not, and when present thickness could be comprised between 15 and 50 micron. Depending on the number of peaks that are needed, part of the coated small units could be administered without any outer layer, while part with thickness values of 20-22 micron, 30-35 micron and 45-50 micron.

Said thickness values corresponds to an increment in weigh of 4 to 11%.

When expressed as coating amount per unit area, said thickness values may be comprised between 2 and 6 mg/cm². Suitable values could be of 22-2.3 mg/cm², 3.4-3.6 mg/cm², and 5.3-5.4 mg/cm².

Advantageously, to make the coated small units suitable for the second phase of the release, the coating outer layer shall be present and its thickness comprised between 45 and 60 micron.

Said thickness values correspond to an increment in weigh of 10 to 18%, preferably of 12 to 16.5%.

When expressed as coating amount per unit area, said thickness values be comprised between 4.5 and 10 mg/cm², preferably between 5 and 6 mg/cm².

The release profile of the minitablets of the invention has been determined according to the dissolution medium and conditions reported in the paragraph about the general experimental details and methods (Release test).

According to the desired pulsatile profile, and in particular if two or three peaks are desired, the skilled person in the art could mix in suitable amounts different types of minitablets having selected thickness of the outer layer.

In a particular embodiment, the invention is directed to a dosage form comprising a mixture of two different types of minitablets having the composition reported in Table 1, wherein the coating thickness of the outer layer of part of minitablets is comprised between 20 and 22 micron and that of the remaining part is comprised between 45 and 50 micron.

In another particular embodiment, the invention is directed to a dosage form comprising a mixture of two different types of minitablets having the composition reported in Table 1, wherein the coating thickness of the outer layer of part of minitablets is comprised between 30 and 35 micron, and that of the remaining part is comprised between 45 and 50 micron.

In a further particular embodiment, the invention is directed to a dosage form comprising a mixture of two different types of minitablets having the composition reported in Table 1, wherein the coating thickness of the outer layer of part of minitablets is comprised between 20 and 22 micron, and that of the remaining part is comprised between 30 and 35 micron.

Yet, in a further particular embodiment, the invention is directed to a dosage form comprising a mixture of three different types of minitablets having the composition reported in Table 1, wherein the coating thickness of the outer layer of part of minitablets is comprised between 20 and 22 micron, that of another part is comprised between 30 and 35 micron, while that of the remaining part is comprised between 45 and 50 micron.

The invention also provides a process for the preparation of the coated small units as described above in form of minitablets, said process comprising the following steps:
(i) mixing the active ingredient with the filler agent, and with the lubricant/glidant excipient to form a mixture;
(ii) compressing the mixture obtained in step (i) to form the minitablets;
(iii) coating the minitablets with the inner layer, and
(iv) optionally, coating the minitablet of step iii), with a further outer layer to obtain double coated minitablets, and
(v) drying the coated minipellets.

Optionally, the mixture of step i) is wet granulated in order to obtain granules suitable to be tableted.

Apparatus and conditions for compaction and granulation are known to the skilled person in the art. Therefore, the operating parameters shall be adjusted according to its knowledge.

For example, typical process parameters for deferiprone are reported in Table 3 of Example 1.

In an alternative embodiment, when the small coated units are in form of pellets, the invention provides a process for the preparation of the coated pellets, said process comprising the following steps:
i) mixing the active ingredient with the filler agent, and with the lubricant/glidant excipient to form a mixture;
ii) wetting the mixture of step i) with a suitable liquid binder;
iii) extruding the wet mass of step ii) through a die with holes to form cylindrical extrudates;
iv) spheronizing the extudates of step iii) to obtain wet pellets;
v) drying the obtained pellets:
vi) coating the pellets with the inner layer;
vii) optionally coating the pellets of step vi) with a further outer layer, and
viii) drying the coated pellets.

Apparatus and conditions for manufacturing pellets are known to the skilled person in the art.

Therefore, the operating parameters shall be adjusted according to its knowledge.

Typically, the minitablets have a diameter of 2.0-3.0 mm, preferably 2.5-2.7 mm, and a height of 2.3-3.2 mm, preferably 2.5-3.0 mm.

Apparatus and conditions for drying the coated formulations are known in the art.

The present disclosure provides dosing regimens useful for the therapeutic use of the pharmaceutical formulations described herein.

Typically, the oral daily dose of deferiprone could range from 75 mg/kg to 100 mg/kg.

The unit dose of deferiprone is typically 1000 mg, but depending on the number of minitablets, different doses could be administered according to the sex, age, weight of the patient.

The claimed dosage forms are useful for the treatment of diseases which cause an overload of iron, or for the prevention and/or treatment of diseases which are caused by an overload of iron.

In some embodiments, the subject in need thereof suffers from iron overload due to transfusional iron overload, or due to diseases such as thalassemia, myelodysplasia, or sickle cell disease.

In some embodiments, the subject in need thereof could suffer from a neurodegenerative disease (e.g., Parkinson's disease, amyotrophic lateral sclerosis (ALS), Huntington's disease, Friedreich's Ataxia, Pantothenate Kinase Associated Neurodegeneration (PKAN), or neurodegeneration with brain iron accumulation (NBIA).

In some embodiments, the subject in need thereof suffers from iron overload that is transfusional iron overload.

In certain aspects, the subject suffers from transfusional iron overload and whose prior chelation therapy is inadequate. In certain aspects, the subject suffers from transfusion iron overload and has a cardiac MRI T2* of 20 ms or less (e.g., 10 ms).

The invention is also directed to a process for filling the pharmaceutical dosage form with the disclosed coated small units.

For example, typical pharmaceutical dosage forms are capsules or sachets.

Apparatus and parameters for filling capsules or sachets are known in the art.

The following examples illustrate the invention without limiting its scope.

### General Experimental Details and Methods

### Preparation of Mini-Tablets

Mini-tablets cores were prepared by compaction of granules obtained by wet granulation. This was necessary to improve the flowability of the powder and the hardness of the mini-tablets. Deferiprone, Avicel PH 101 and silica were mixed in a planetary mixer and a binding aqueous solution of polyvinyl pyrrolidone (PVP) at 5 or 8% w/w concentration was added to the powders. The wet mass was granulated in an oscillating granulator (Erweka, Wet granulator FGS) with a screen size of 1000 μm. Granules were dried in oven at 40 °C overnight. Magnesium stearate (0.5%) and Explotab® CLV (2.5%) were added to granules and compacted into mini-tablets by means a rotary tablet press (Officine Meccaniche Ronchi, AM8S), equipped with concave punches (diameter 2.5 mm, curvature radius 2.5 mm). Compression force was set at 6-7 kN. The mini-tablets produced had a mass and height of approximately 12 mg and 2.2 mm, respectively, and friability <1%. Where indicated, tracer-containing mini-tablet of similar size and physical characteristics and having the following composition were used: acetaminophen (80%), Kollidon®VA64, (2%), Avicel®PH101 (12.5%), Explotab® CLV (4.5%), colloidal silica (0.5%) and magnesium stearate (0.5%). These were prepared by direct compression of the powder mixture.

### Coating of Mini-Tablets

Application of the inner layer consisting of:
HPMC: the process was carried out by fluid bed equipment and rotor insert was employed. Coating formulation and experimental conditions are reported on a case-by-case basis in the corresponding tables. The process was conducted until 250 μm layer thickness was achieved, approximately requiring 11-16 hours depending on the batch size.
L-HPC: the process was carried out by powder layering in fluid bed equipment with rotor insert with tangential spraying gun. Methocel® E5 5% w/v aqueous solution was used as a binding solution.

Application of the outer rupturable film (Eudragit® NE 30D or Surelease®+Explotab® CLV): units provided with HPMC layer were coated by spraying a commercial dispersion of Eudragit® NE 30D or Surelease® according to composition and experimental conditions reported on a case-by-case basis in the corresponding tables. The process was carried out in fluid bed equipment (Mini-Glatt, Glatt GmbH, Germany) with bottom spray configuration and Wurster insert. In order to prepare units at different level of coating, samples were collected at predetermined times during the process.

### Characterization of Coated Mini-Tablets

Units were characterized for weight (m), height (h) and diameter (d).

Weight gain was calculated according to the formula:

$$wg \% = \frac{{m}_{coated} - {m}_{uncoated}}{{m}_{uncoated}} \times 100$$

where m_{coated} and m_{uncoated} are the mean weight values of n=100 units.

Thickness of both the coating layers was calculated according to the formula:

$$thickness = \frac{\frac{{h}_{coated} - {h}_{uncoated}}{2} + \frac{{d}_{coated} - {d}_{uncoated}}{2}}{2} * 1000$$

where h_{coated} and h_{uncoated} and d_{coated} and d_{uncoated} are the mean height and diameter values of n=20 units.

Coating mass per unit area was calculated according to the formula:

$$Coating mass / unit area \frac{{m}_{coated} - {m}_{uncoated}}{2 π {r}_{uncoated} {h}_{uncoated} + 2 π {r}_{uncoated}^{2}}$$

where r_{uncoated}=½d_{uncoated}

### Release Test

Release tests were performed by an adapted three-position disintegration testing apparatus. This was selected to overcome the possible adhesion of the hydrated HPMC coating to the vessels of a paddle dissolution apparatus. Each unit was inserted into a basket-rack assembly so that only one of the 6 available tubes was filled. During the test, every basket-rack assembly moved at a rate of 31 cycles/min in a separate vessel containing 800 ml of water t=37±0.5 °C. Fluid samples were withdrawn automatically at predetermined time points and deferiprone or the tracer (acetaminophen) was quantified spectrophotometrically (Lambda 25, Perkin Elmer, Italy).

### Example 1—Preparation and Characterization of the Coated Minitablets According to the Invention

According to Chronotopic® system design, mini-tablet cores were double coated to provide a first inner HPMC layer of 250 m thickness and an outer layer consisting of an insoluble polymer and generally containing Explotab® CLV (EXP) as film pore former.

In an initial stage of this evaluation, Eudragit® NE was selected as the coating polymer for the outer layer, then Ethylcellulose (EC) was preferred.

However, the amount in which Eudragit® NE could be applied was poorly drivable within the coating operations in use, thus a meaningful differentiation of the performances of the various coated units could not be achieved.

For the production of the mini-tablets containing Explotab® CLV in the outer layer, PVP concentration of the binder solution as well as the concentration of Methocel®E50 in the coating formulation were adjusted to improve the outcome of spraying.

The composition of the minitablets before drying is reported in Table 2, while the process parameters and their characterization are reported in Tables 3 and 4, respectively.

TABLE 2
Composition of the minitablets of the invention after drying

| Mini-tablet core composition | % w/w |
|---|---|
| Deferiprone | 81.4 |
| Avicel PH 101 | 14.2 |
| Silica | 0.1 |
| PVP | 1.3 |
| Magnesium stearate | 0.5 |
| Explotab ® CLV | 2.4 |

| Coating formulation (% w/w) | HPMC layer | EC/EXP layer |
|---|---|---|
| Methocel E50 | 90 | — |
| PEG 400 | 10 | — |
| Surelease ® | — | 95 |
| Explotab CLV | — | 5 |

TABLE 3
Process parameters

| Process parameters | HPMC layer | EC/EXP layer |
|---|---|---|
| Equipment type | Glatt GPC1 | Mini-Glatt |
| | tangential spray | bottom spray |
| Batch size (g) | 600 | 25 |
| Inlet air temperature (°C) | 65 | 62 |
| Product temperature (°C) | 48 | 38/42 |
| Coater airflow (m³/hr) | 90 | 27/29 |
| Atomization pressure (bar) | 1.5 | 0.8 |
| Liquid spraying rate (g/min) | 8-10 | 0.3 |
| Curing | | 40 °C for |
| | | 24 h |

TABLE 4
Characterization of the minitablets of the invention

| | Uncoated | | | | | | |
|---|---|---|---|---|---|---|---|
| | (mini-tablet | HPMC | | | | | |
| | core) | coated | EC/EXP5 coated | | | | |
| | C | C0 | C3 | C6 | C9 | C11 | C17 |
| Weight^{#} | 12.9 | 19.33 | 20.21 | 20.68 | 21.43 | 21.81 | 22.52 |
| (mg) | | | | | | | |
| Weight | | | 4.55 | 6.98 | 10.86 | 12.83 | 16.50 |
| gain after | | | | | | | |
| coating (%) | | | | | | | |
| Height* | 2.517 ± 0.024 | 2.966 ± 0.031 | 3.021 ± 0.024 | 3.049 ± 0.028 | 3.083 ± 0.077 | 3.078 ± 0.05 | 3.112 ± 0.034 |
| (mm) | | | | | | | |
| Diameter* | 2.217 ± 0.035 | 2.74 ± 0.029 | 2.771 ± 0.062 | 2.791 ± 0.045 | 2.846 ± 0.078 | 2.784 ± 0.046 | 2.83 ± 0.034 |
| (mm) | | | | | | | |
| Coating | — | 250 | 21 | 33 | 47 | 52 | 59 |
| layer | | | | | | | |
| thickness* | | | | | | | |
| (μm) | | | | | | | |
| Coating | | 23.48 | 2.24 | 3.43 | 5.34 | 6.31 | 8.11 |
| amount per | | | | | | | |
| unit area | | | | | | | |
| (mg/cm²) | | | | | | | |

*mean ± sd n = 20
^{#}mean n = 100

The described mini-tablets show satisfactory technological characteristics and release performance meeting the expected pulsatile behaviour.

The outer film thickness, brings a fundamental value to the mechanism of release control. Indeed, the delay performance is directly correlated to this parameter. As it can be appreciated from FIG. 4, C0, C3 and C6 minitablets displayed in vitro lag phases potentially suitable for determining in vivo the pursued, distinct release pulses, while C9, C11 and C17 minitablets would be expected to exhibit a delayed release matching the scope of their use in deferiprone oral therapy.

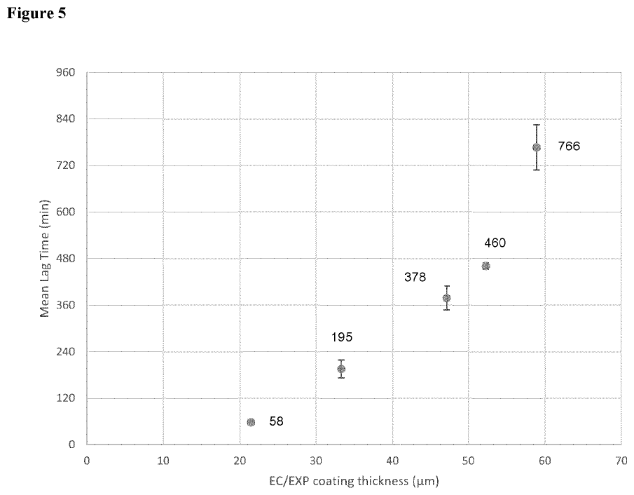

From FIG. 5, it could be appreciated how the lag time of the minitablets depend on the thickness of the outer layer.

### Example 2—Preparation of the Uncoated Pellets According to the Invention

Small units in form of pellets with compositions reported in Table 5 were prepared by extrusion/spheronization.

TABLE 5
Composition of the uncoated pellets.

| | Composition (% w/w) | | | | |
|---|---|---|---|---|---|
| | | Avicel | Vivapur | Magnesium | |
| Formulation | Deferiprone | CL611 | 101 | stearate | Silica |
| A | 89 | 10.4 | / | 0.5 | 0.1 |
| B | 89 | / | 10.4 | 0.5 | 0.1 |

Deferiprone and excipients were mixed in a planetary mixer and water was added to the powders. The mixture was pushed into the extruder with a screen size of 1000 μm. Experimental conditions of the extrusion and spheronization phases are reported in Tables 6 and 7, respectively. The wet units were dried in static oven at 40 °C overnight.

TABLE 6
Extrusion experimental conditions.

| | Feeder | Extrusion head | Water |
|---|---|---|---|
| | (RPM) | (RPM) | (g) |
| A | 50 | 60 | 44.20 |
| B (batch 1) | 50 | 60 | 43.80 |
| B batch 2 | 50 | 60 | 56.30 |

TABLE 7
Spheronization experimental conditions.

Parameters

| | Speed | |
|---|---|---|
| | (RPM) | Time (sec) |
| A | 300 | 60 |
| | 450 | 300 |
| B batch 1 | 300 | 150 |
| B batch 2 | 300 | 120 |

It is expected that when the pellets are coated as reported in Example 1, they would show the same release profile of the coated minitablets.

1: A pharmaceutical dosage form suitable for providing a pulsatile release profile, said form comprising coated small units, wherein each unit in turn comprises an active ingredient, a filler, a lubricant and/or a glidant and it is coated with an inner layer whereby one part of the coated small units releases a fraction of the active ingredient in a time between 10 minutes and 8 hours, while the remaining part releases the remaining active ingredient in a time between 6 hours and 24 hours.

2: The pharmaceutical dosage form according to claim 1, wherein at least the coated small units releasing the fraction of the active ingredient in a time between 10 minutes and 8 hours are further covered with an outer layer.

3: The pharmaceutical dosage form according to claim 1, wherein the coated small unit comprises the active ingredient in an amount between 75% and 90%, a filler in an amount between 10% and 24%, and a lubricant and/or glidant in an amount of 0.5 to 1.0%, all the amounts calculated by weight based on a total weight of the uncoated unit; and
wherein the inner layer comprises a swellable hydrophilic polymer and a plasticizer, and an outer layer, when present, comprises a film-forming insoluble polymer and an agent with channeling properties.

4: The pharmaceutical dosage form according to claim 1, wherein the active ingredient is deferiprone.

5: The pharmaceutical dosage form according to claim 1, wherein the filler is a mixture of a diluent and a superdisintegrant agent in any suitable ratio.

6: The pharmaceutical dosage form according to claim 5, wherein the ratio between diluent and the superdisintegrant agent is between 85:15 and 75:25 w/w.

7. (canceled)

8: The pharmaceutical dosage form according to claim 5, wherein the diluent is selected from the group consisting of calcium carbonate, dibasic calcium phosphate, tribasic calcium phosphate, calcium sulfate, microcrystalline cellulose, powdered cellulose, dextrans, dextrins, dextrose, fructose, kaolin, lactose, mannitol, sorbitol, starch, pregelatinized starch, sucrose, and alpha-lactose monohydrate.

9. (canceled)

10: The pharmaceutical dosage form according to claim 5, wherein the superdisintegrant agent is selected from the group consisting of croscarmellose sodium, crospovidone and sodium starch glycolate.

11-12. (canceled)

13: The pharmaceutical dosage form according to claim 1, wherein the lubricant is at least one selected from the group consisting of magnesium stearate, calcium stearate, stearic acid, and sodium stearyl fumarate.

14: The pharmaceutical dosage form according to claim 1, wherein the glidant is at least one selected from the group consisting of colloidal silicon dioxide, starch and talc.

15: The pharmaceutical dosage form according to claim 1, wherein the small unit comprises a mixture of colloidal silicon dioxide and magnesium stearate in a ratio of 10:90 to 20:80 w/w.

16. (canceled)

17: The pharmaceutical dosage form according to claim 1, wherein the inner layer of the coating comprises a swellable hydrophilic polymer.

18: The pharmaceutical dosage form according to claim 17, wherein the inner layer comprises a hydroxypropyl methylcellulose and a plasticizer.

19: The pharmaceutical dosage form according to claim 18, wherein the plasticizer is at least one selected from the group consisting of diethyl phthalate, a citrate ester, polyethylene glycol, glycerol, an acetylated glyceride, an acetylated citrate ester, dibutyl sebacate, and castor oil.

20-22. (canceled)

23: The pharmaceutical dosage form according to claim 2, wherein the outer layer of the coating comprises a film-forming insoluble polymer and a superdisintegrant agent.

24-26. (canceled)

27: The pharmaceutical dosage form according to claim 1, wherein the small unit to be coated is in a form of minitablets or pellets.

28: A process for the preparation of the pharmaceutical dosage form according to claim 27, said process comprising:
i) mixing the active ingredient with the filler, and with the lubricant/glidant excipient to form a mixture;
ii) compressing the mixture obtained in i) to form the minitablets;
iii) coating the minitablets with the inner layer, and
iv) optionally, coating the minitablets of iii), with a further outer layer to obtain double coated minitablets, and
v) drying the coated minitablets.

29: The process according to claim 28, wherein the mixture of i) is wet granulated.

30: A process for the preparation of the pharmaceutical dosage form according to claim 27, said process comprising:
i) mixing the active ingredient with the filler, and with the lubricant/glidant excipient to form a mixture;
ii) wetting the mixture of i) with a suitable liquid binder to obtain a wet mass;
iii) extruding the wet mass of ii) through a die with holes to form cylindrical extrudates;
iv) spheronizing the cylindrical extudates of iii) to obtain wet pellets;
v) drying the wet pellets to obtain dry pellets:
vi) coating the dry pellets with the inner layer;
vii) optionally coating the pellets of vi) with a further outer layer, and
viii) drying the coated pellets.

31. (canceled)

32: A method of treating a disease that causes an overload of iron comprising administering the pharmaceutical dosage form according to claim 1 to a subject in need thereof, wherein the pharmaceutical dosage form comprises deferiprone as an active ingredient, and wherein the pharmaceutical dosage form is in a form of capsules or sachets.

33-34. (canceled)
